(19) United States
(12) Patent Application Publication
(10) Pub. No.: US 2008/0207953 A1
(43) Pub. Date: Aug. 28, 2008

(54) Catalyst and Method for Hyrogenating Carbonyl Compounds

(75) Inventors: Christophe Houssin, Mannheim (DE); Henrik Junicke, Mannheim (DE); Ulrich Muller, Neustadt (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/994,500

(22) PCT Filed: Jul. 6, 2006
(86) PCT No.: PCT/EP2006/063958

§ 371 Date: Jan. 3, 2008

(30) Foreign Application Priority Data
Jul. 13, 2005 (EP) 10 2005 032 726.5

(51) Int. Cl. C07C 47/00; B01J 21/18

(52) U.S. Cl. 568/420; 502/184

(57) ABSTRACT

Process for hydrogenating an organic compound having at least one carbonyl group, in which the organic compound is contacted, in the presence of hydrogen, with a shaped body which is producible in a process in which
(i) an oxidic material comprising copper oxide and aluminum oxide and at least one of the oxides of iron, lanthanum, tungsten, molybdenum, titanium, zirconium, tin or manganese, and if appropriate additionally tin oxide and/or manganese oxide, is provided,
(ii) pulverulent metallic copper, copper flakes, pulverulent cement, graphite or a mixture thereof are added to the oxidic material,
(iii) the mixture resulting from (ii) is shaped to a shaped body and
(iv) the shaped body is treated with water or steam.

The present invention relates to a process for hydrogenating organic compounds which have at least one carbonyl group using a catalyst which, among other features, consists of copper oxide, aluminum oxide and at least one of the oxides of iron, lanthanum, tungsten, molybdenum, titanium, zirconium, tin or manganese, and treatment with boiling water and/or steam gives rise to a catalyst with high selectivity and simultaneously high stability. In the course of its production, copper powder, copper flakes or cement may additionally be added.

The catalytic hydrogenation of carbonyl compounds, for example carboxylic acids or carboxylic esters, is assuming an important position in the production streams of the commodity chemicals industry.

The catalytic hydrogenation of carbonyl compounds, for example carboxylic esters, is carried out almost exclusively in fixed bed reactors in industrial processes. The fixed bed catalysts used, in addition to catalysts of the Raney type, are in particular supported catalysts, for example copper, nickel or noble metal catalysts.

U.S. Pat. No. 3,923,694 describes, for example, a catalyst of the copper oxide/zinc oxide/aluminum oxide type. The disadvantage of this catalyst is that it is not sufficiently mechanically stable during the reaction and therefore decomposes relatively rapidly. This results in a loss of activity and a buildup of differential pressure over the reactor as a result of the decomposing shaped catalyst bodies. The consequence is that the plant has to be shut down prematurely.

DE 198 09 418.3 describes a process for catalytically hydrogenating a carbonyl compound in the presence of a catalyst which comprises a support which comprises primarily titanium dioxide, and, as the active component, copper or a mixture of copper with at least one of the metals selected from the group of zinc, aluminum, cerium, a noble metal and a metal of transition group VIII, the copper surface area being not more than 10 m²/g. Preferred support materials are mixtures of titanium dioxide with aluminum oxide or zirconium oxide or aluminum oxide and zirconium oxide. In a preferred embodiment, the catalyst material is shaped with addition of metallic copper powder or copper flakes.

DE-A 195 05 347 describes, in quite general terms, a process of catalyst tablets with high mechanical strength, in which a metal powder or a powder of a metal alloy is added to the material to be tableted. The metal powders added include aluminum powder or copper powder or copper flakes. In the case of the addition of aluminum powder, however, a shaped body obtained with a copper oxide/zinc oxide/aluminum oxide catalyst has a worse side crushing strength than a shaped body which was prepared without addition of aluminum powder, and the inventive shaped body exhibited, when it was used as a catalyst, a poorer conversion activity than catalysts which were produced without addition of aluminum powder. Likewise disclosed there is a hydrogenation catalyst composed of NiO, ZrO₂, MoO₃ and CuO, to which materials including copper powder have been added in the course of production. However, this document does not make any statements on the selectivity or the activity.

DE 256 515 describes a process for preparing alcohols from synthesis gas, in which catalysts based on Cu/Al/Zn are used, which are obtained by joint grinding and pilling of metallic copper powder or copper flakes. The main emphasis in the process described is on the preparation of mixtures of C₁ to C₅ alcohols, a process being selected in which the reaction reactor comprises, in the upper third of the bed, a catalyst which has a higher proportion of copper powder or copper flakes, and, in the lower third, comprises a catalyst which has a lower proportion of copper powder or copper flakes.

JP-A 50-99987 describes the increase in the mechanical stability of specific Raney catalysts which may be copper-based by water or steam treatment. SU-A 728 908 discloses the hardening of aluminum-copper-zinc catalysts for methanol synthesis by water treatment. Neither document makes any statements on the selectivity or activity.

It was an object of the present invention to provide a process and a catalyst which do not have the disadvantages of the prior art and provide processes for catalytically hydrogenating carbonyl compounds and also catalysts, the catalysts having both high mechanical stability and high hydrogenation activity and selectivity.

It has been found that the simultaneous precipitation of copper and of an aluminum compound and also, if appropriate, additionally a compound of iron, lanthanum, tungsten, molybdenum, titanium, zirconium, tin and/or manganese, and the subsequent drying, calcining, tableting, and the addition of metallic copper powder, copper flakes or cement powder or graphite or a mixture affords a catalyst which leads, by virtue of a water and/or steam treatment, both to high activities and selectivities, and to a high stability of the shaped body which is used as a catalyst.

Accordingly, the present invention relates to a process for hydrogenating an organic compound having at least one carbonyl group, in which the organic compound is contacted, in the presence of hydrogen, with a shaped body which is producible in a process in which
(i) an oxidic material comprising copper oxide and aluminum oxide and at least one of the oxides of iron, lanthanum, tungsten, molybdenum, titanium, zirconium, tin or manganese is provided,
(ii) pulverulent metallic copper, copper flakes, pulverulent cement, graphite or a mixture thereof may be added to the oxidic material,
(iii) the mixture resulting from (ii) is shaped to a shaped body and
(iv) the shaped body is treated with boiling water and/or steam.

Iron oxide is understood to mean iron(III) oxide.

In preferred embodiments, the inventive shaped bodies are used in the form of unsupported catalysts, impregnated catalysts, coated catalysts and precipitation catalysts.

The catalyst used in the process according to the invention has the feature that the copper active component, the aluminum component and the component of at least one of the oxides of iron, lanthanum, tungsten, molybdenum, titanium, zirconium, tin or manganese are preferably precipitated with a sodium carbonate solution simultaneously or successively, subsequently dried, calcined, tableted and calcined once more.

In particular, the following precipitation method is useful:
A) A copper salt solution, an aluminum salt solution and a solution of a salt of iron, lanthanum, tungsten, molybdenum, titanium, zirconium, tin or manganese, or a solution comprising copper, aluminum and a salt of iron, lanthanum, tungsten, molybdenum, titanium, zirconium, tin or manganese, is precipitated with a sodium carbonate solution in parallel or successively. The precipitated material is subsequently dried and, if appropriate, calcined.
B) Precipitation of a copper salt solution and of a solution of a salt of iron, lanthanum, tungsten, molybdenum, titanium, zirconium, tin or manganese, or of a solution comprising copper salt and at least one salt of iron, onto a prefabricated aluminum oxide support. In a particularly preferred embodiment, this is present in the form of a powder in an aqueous suspension. However, the support material may also be present in the form of spheres, extrudates, spall or tablets.
B1) In one embodiment (I), a copper salt solution and a solution of a salt of iron, lanthanum, tungsten, molybdenum, titanium, zirconium, tin or manganese, or a solution comprising copper salt and a salt of iron, lanthanum, tungsten, molybdenum, titanium, zirconium, tin or manganese, is preferably precipitated with sodium carbonate solution. The initial charge used is an aqueous suspension of the aluminum oxide support material.

Precipitated solids which result from A) or B) are typically filtered and preferably washed to free them of alkali, as described, for example, in DE 198 09 418.3.

Both the end products from A) and from B) are dried at temperatures of from 50 to 150° C., preferably at 120° C., and subsequently, if appropriate, calcined preferably for 2 hours at generally from 200 to 600° C., in particular at from 300 to 500° C.

The starting substances used for A) and/or B) may in principle be all Cu(l) and/or Cu(II) salts soluble in the solvents used in the application, for example nitrates, carbonates, acetates, oxalates or ammonium complexes, analogous aluminum salts and salts of iron. For processes according to A) and B), particular preference is given to using copper nitrate.

In the process according to the invention, the above-described dried and, if appropriate, calcined powder is processed preferably to tablets, rings, ring tablets, extrudates, honeycombs or similar shaped bodies. For this purpose, all suitable processes from the prior art are conceivable. Particular preference is given to using a shaped catalyst body or a catalyst extrudate with a diameter d and a height h<5 mm, catalyst spheres with a diameter d of <6 mm or catalyst honeycombs with a cell diameter r_{z}<5 mm.

The composition of the oxidic material is generally such that the proportion of copper oxide is in the range from 40 to 90% by weight, the proportion of oxides of iron, lanthanum, tungsten, molybdenum, titanium, zirconium, tin or manganese is in the range from 0 to 50% by weight, and the proportion of aluminum oxide is in the range of up to 50% by weight, based in each case on the total weight of the sum of the abovementioned oxidic constituents, these three oxides together constituting at least 80% by weight of the oxidic material after calcination, cement not being included in the oxidic material in the above sense.

In a preferred embodiment, the present invention therefore relates to a process as described above, wherein the oxidic material comprises
(a) copper oxide with a proportion in the range of 50≦x≦80% by weight, preferably 55≦x≦75% by weight,
(b) aluminum oxide with a proportion in the range of 15≦y≦35% by weight, preferably 20≦y≦30% by weight and
(c) at least one of the oxides of iron, lanthanum, tungsten, molybdenum, titanium, zirconium, tin or manganese with a proportion in the range of 1≦z≦30% by weight, preferably 2≦z≦25% by weight,

based in each case on the total weight of the oxidic material after calcination, where: 80≦x+y+z≦100, especially 95≦+y+z≦100.

The inventive process and the inventive catalysts are notable in that, by virtue of the treatment of the shaped body with boiling water and/or steam, a high stability of the shaped body which is used as a catalyst is achieved, and the hydrogenation activity and selectivity of the catalyst is simultaneously increased.

For the water treatment, the shaped body which has been dried and calcined as described above is covered in an amount of water or of an aqueous-alcoholic solution with a C₁- to C₄ alcohol such as methanol, ethanol or butanol which is sufficient to fully cover the catalyst. The aqueous-alcoholic solutions have a maximum alcohol concentration of 30% by weight. When water is used, the pH is adjusted to from 4 to 9, preferably to from 6 to 8.5, with the aid of mineral acids such as nitric acid, sulfuric acid or hydrochloric acid or sodium carbonate or sodium hydroxide solution. The catalysts are treated with water or the aqueous-alcoholic solution at from 100 to 140° C. and a pressure of from 1 to 30 bar, preferably at from 1 to 3 bar, for from 1 to 48 h, preferably from 5 to 20 h.

The steam treatment may be carried out with 100% steam, with vapor mixtures composed of steam and inert gases, for example nitrogen, with a proportion of the inert gas of up to 90% by weight, and/or with vapors of compounds in which water is formed under the reaction conditions of the steam treatment, for example the C₁ to C₄ alcohols such as methanol, ethanol or butanol, with an alcohol proportion of not more than 90% by weight. Preference is given to carrying out the steam treatment with pure steam.

The catalyst bodies are treated with steam at from 100 to 300° C., preferably at from 100 to 150° C., generally at standard pressure, but an elevated pressure of from 1 to 20 bar, preferably from 1 to 2 bar, is also possible. The steam treatment will generally proceed for at least 1 h; preference is given to from 10 to 48 h of treatment time.

After the water and/or steam treatment, the shaped catalyst body is dried again at temperatures of 120° C., preferably for 2 h at generally from 5 to 300° C., and calcined if appropriate.

In general, pulverulent copper, copper flakes or pulverulent cement or graphite or a mixture thereof is added to the oxidic material in the range from 1 to 40% by weight, preferably in the range from 2 to 20% by weight and more preferably in the range from 3 to 10% by weight, based in each case on the total weight of the oxidic material.

The cement used is preferably an alumina cement. The alumina cement more preferably consists substantially of aluminum oxide and calcium oxide, and more preferably consists of from about 75 to 85% by weight of aluminum oxide and from about 15 to 25% by weight of calcium oxide. In addition, it is also possible to use a cement based on magnesium oxide/aluminum oxide, calcium oxide/silicon oxide and calcium oxide/aluminum oxide/iron oxide.

In particular, the oxidic material may have, in a proportion of at most 10% by weight, preferably at most 5% by weight, based on the total weight of the oxidic material, of at least one further component which is selected from the group consisting of the elements Re, Fe, Ru, Co, Rh, Ir, Ni, Pd and Pt.

In a further preferred embodiment of the process according to the invention, graphite is added to the oxidic material before the shaping to the shaped body in addition to the copper powder, the copper flakes or the cement powder or the mixture thereof. Preference is given to adding sufficient graphite that the shaping to a shaped body can be carried out better. In a preferred embodiment, from 0.5 to 5% by weight of graphite, based on the total weight of the oxidic material, are added. It is immaterial whether graphite is added to the oxidic material before or after or simultaneously with the copper powder, the copper flakes or the cement powder or the mixture thereof.

Accordingly, the present invention also relates to a process as described above, wherein graphite is added to the oxidic material or to the mixture resulting from (ii) in a proportion in the range from 0.5 to 5% by weight based on the total weight of the oxidic material.

In a preferred embodiment, the present invention therefore also relates to a shaped body, treated with boiling water and/or steam and comprising

an oxidic material which comprises
(a) copper oxide with a proportion in the range of 50≦x≦80% by weight, preferably 55≦x≦75% by weight,
(b) aluminum oxide with a proportion in the range of 15≦y≦35% by weight, preferably 20≦y≦30% by weight and
(c) at least one of the oxides of iron, lanthanum, tungsten, molybdenum, titanium, zirconium, tin or manganese with a proportion in the range of 1≦z≦30% by weight, preferably from 2 to 25% by weight,

based in each case on the total weight of the oxidic material after calcination, where: 80≦x+y+z≦100, especially 95≦x+y+z≦100,

metallic copper powder, copper flakes or cement powder or a mixture thereof with a proportion in the range from 1 to 40% by weight based on the total weight of the oxidic material and

graphite with a proportion of from 0.5 to 5% by weight based on the total weight of the oxidic material,

the sum of the proportions of oxidic material, metallic copper powder, copper flakes or cement powder or a mixture thereof and graphite adding up to at least 95% by weight of the shaped body.

After addition of the copper powder, of the copper flakes or of the cement powder or of the mixture thereof and, if appropriate, graphite to the oxidic material, the shaped body obtained after the shaping is, if appropriate, calcined at least once over a period of generally from 0.5 to 10 h, preferably from 0.5 to 2 hours. The temperature in this at least one calcination step is generally in the range from 200 to 600° C., preferably in the range from 250 to 500° C. and more preferably in the range from 270 to 400° C.

In the case of shaping with cement powder, it may be advantageous to moisten the shaped body obtained before the calcination with water and subsequently to dry it.

In the case of use as a catalyst in the oxidic form, the shaped body, before charging with the hydrogenation solution, is pre-reduced with reducing gases, for example hydrogen, preferably hydrogen-inert gas mixtures, especially hydrogen/nitrogen mixtures, at temperatures in the range from 100 to 500° C., preferably in the range from 150 to 350° C. and in particular in the range from 180 to 200° C. Preference is given to using a mixture having a hydrogen content in the range from 1 to 100% by volume, more preferably in the range from 1 to 50% by volume.

In a preferred embodiment, the inventive shaped body, before use as a catalyst, is activated in a manner known per se by treatment with reducing media. The activation is effected either beforehand in a reduction oven or after installation in the reactor. When the reactor has been activated beforehand in the reduction oven, it is installed into the reactor and charged with the hydrogenation solution directly under hydrogen pressure.

The preferred field of use of the shaped bodies prepared by the process according to the invention is the hydrogenation of organic compounds having carbonyl groups in a fixed bed. Other embodiments, for example the fluidized reaction with catalyst material in upward and downward motion, are, however, likewise possible. The hydrogenation may be carried out in the gas phase or in the liquid phase. Preference is given to carrying out the hydrogenation in the liquid phase, for example in trickle mode or liquid phase mode.

Working in trickle mode allows the liquid reactant comprising the carbonyl compound to be hydrogenated, in the reactor which is under hydrogen pressure, to trickle over the catalyst bed arranged therein, a thin liquid film being formed on the catalyst. In contrast, when working in liquid phase mode, hydrogen gas is introduced into the reactor flooded with the liquid reaction mixture, the hydrogen passing through the catalyst bed in ascending gas bubbles.

In one embodiment, the solution to be hydrogenated is pumped in straight pass through the catalyst bed. In another embodiment of the process according to the invention, a portion of the product is drawn off continuously as a product stream after passing through the reactor and, if appropriate, passed through a second reactor as defined above. The other portion of the product is fed back to the reactor together with fresh reactant comprising the carbonyl compound. This procedure is referred to below as circulation mode.

When, as an embodiment of the process according to the invention, trickle mode is selected, preference is given here to circulation mode. Preference is further given to working in circulation mode with use of a main reactor and postreactor.

The process according to the invention is suitable for hydrogenating carbonyl compounds, for example aldehydes and ketones, carboxylic acids, carboxylic esters or carboxylic anhydrides, to the corresponding alcohols, preference being given to alipatic and cycloaliphatic, saturated and unsaturated carbonyl compounds. In the case of aromatic carbonyl compounds, undesired by-products may be formed by hydrogenation of the aromatic ring. The carbonyl compounds may bear further functional groups such as hydroxyl or amino groups. Unsaturated carbonyl compounds are generally hydrogenated to the corresponding saturated alcohols. The term “carbonyl compounds” as used in the context of the invention comprises all compounds which have a C═O group, including carboxylic acids and their derivatives. It will be appreciated that it is also possible to hydrogenate mixtures of two or more than two carbonyl compounds together. It is also possible for the individual carbonyl compound to be hydrogenated to comprise more than one carbonyl group.

Preference is given to using the process according to the invention for hydrogenating aliphatic aldehydes, hydroxy aldehydes, ketones, acids, esters, anhydrides, lactones and sugars.

Preferred aliphatic aldehydes are branched and unbranched, saturated and/or unsaturated aliphatic C₂-C₃₀ aldehydes, as are obtainable, for example, by oxo synthesis from linear or branched olefins with internal or terminal double bonds. It is also possible to hydrogenate oligomeric compounds which also comprise more than 30 carbonyl groups.

Examples of aliphatic aldehydes include:

formaldehyde, propionaldehyde, n-butyraldehyde, isobutyraldehyde, valeraldehyde, 2-methylbutyraldehyde, 3-methylbutyraldehyde (isovaleraldehyde), 2,2-dimethyl-propionaldehyde (pivalaldehyde), caproaldehyde, 2-methylvaleraldehyde, 3-methylvaleraldehyde, 4-methylvaleraldehyde, 2-ethylbutyraldehyde, 2,2-dimethyl-butyraldehyde, 3,3-dimethylbutyraldehyde, caprylaldehyde, capraldehyde, glutaraldehyde.

In addition to the short-chain aldehydes mentioned, long-chain aliphatic aldehydes are also especially suitable, as can be obtained, for example, by oxo synthesis from linear α-olefins.

Particular preference is given to enalization products, for example 2-ethylhexenal, 2-methylpentenal, 2,4-diethyloctenal or 2,4-dimethylheptenal.

Preferred hydroxy aldehydes are C₃-C₁₂ hydroxy aldehydes, as are obtainable, for example, by aldol reaction from aliphatic and cycloaliphatic aldehydes and ketones with themselves or formaldehyde. Examples are 3-hydroxypropanal, dimethylolethanal, trimethylolethanal (pentaerythrital), 3-hydroxybutanal (acetaldol), 3-hydroxy-2-ethylhexanal (butylaldol), 3-hydroxy-2-methylpentanal (propionaldol), 2-methylol-propanal, 2,2-dimethylolpropanal, 3-hydroxy-2-methylbutanal, 3-hydroxypentanal, 2-methylolbutanal, 2,2-dimethylolbutanal, hydroxypivalaldehyde. Particular preference is given to hydroxypivalaldehyde (HPA) and dimethylolbutanal (DMB).

Preferred ketones are acetone, butanone, 2-pentanone, 3-pentanone, 2-hexanone, 3-hexanone, cyclohexanone, isophorone, methyl isobutyl ketone, mesityl oxide, acetophenone, propiophenone, benzophenone, benzalacetone, dibenzalacetone, benzalacetophenone, 2,3-butanedione, 2,4-pentanedione, 2,5-hexanedione and methyl vinyl ketone.

It is also possible to convert carboxylic acids and derivatives thereof, preferably those having 1-20 carbon atoms. The following should be mentioned in particular:

carboxylic acids, for example formic acid, acetic acid, propionic acid, butyric acid, isobutyric acid, n-valeric acid, trimethylacetic acid (“pivalic acid”), caproic acid, enanthic acid, caprylic acid, capric acid, lauric acid, myristic acid, palmitic acid, stearic acid, acrylic acid, methacrylic acid, oleic acid, elaidic acid, linoleic acid, linolenic acid, cyclohexanecarboxylic acid, benzoic acid, phenylacetic acid, o-toluic acid, m-toluic acid, p-toluic acid, o-chlorobenzoic acid, p-chlorobenzoic acid, o-nitrobenzoic acid, p-nitrobenzoic acid, salicylic acid, p-hydroxybenzoic acid, anthranilic acid, p-amino-benzoic acid, oxalic acid, malonic acid, succinic acid, glutaric acid, adipic acid, pimelic acid, suberic acid, azelaic acid, sebacic acid, maleic acid, fumaric acid, phthalic acid, isophthalic acid, terephthalic acid;

carboxylic esters, for example the C₁-C₁₀-alkyl esters of the abovementioned carboxylic acids, especially methyl formate, ethyl acetate, butyl butyrate, dialkyl phthalates, dialkyl isophthalates, dialkyl terephthalates, dialkyl adipates, dialkyl maleates, for example the dimethyl esters of these acids, methyl (meth)acrylate, butyrolactone, caprolactone and polycarboxylic esters, for example polyacrylic and polymethacrylic esters and their co-polymers and polyesters, for example polymethyl methacrylate, terephthalic esters and other industrial plastics, in which case hydrogenolyses, i.e. the conversion of esters to the corresponding acids and alcohols, are carried out in particular;

fats;

carboxylic anhydrides, for example the anhydrides of the abovementioned carboxylic acids, especially acetic anhydride, propionic anhydride, benzoic anhydride and maleic anhydride;

carboxamides, for example formamide, acetamide, propionamide, stearamide, terephthalamide.

It is also possible to convert hydroxy carboxylic acids, for example lactic acid, malic acid, tartaric acid or citric acid, or amino acids, for example glycine, alanine, proline and arginine, and peptides.

Particularly preferred organic compounds to be hydrogenated are saturated or unsaturated carboxylic acids, carboxylic esters, carboxylic anhydrides or lactones or mixtures of two or more thereof.

Accordingly, the present invention also relates to a process as described above, wherein the organic compound is a carboxylic acid, a carboxylic ester, a carboxylic anhydride or a lactone.

Examples of these compounds include maleic acid, maleic anhydride, succinic acid, succinic anhydride, adipic acid, 6-hydroxycaproic acid, 2-cyclododecylpropionic acid, the esters of the aforementioned acids, for example methyl, ethyl, propyl or butyl esters. Further examples are y-butyrolactone and caprolactone.

In a very particularly preferred embodiment, the present invention relates to a process as described above, wherein the organic compound is adipic acid or an adipic ester.

The carbonyl compound to be hydrogenated can be fed to the hydrogenation reactor alone or as mixture with the product of the hydrogenation reaction, in which case this can take place in undiluted form or with use of additional solvent. Suitable additional solvents are in particular water, alcohols such as methanol, ethanol and the alcohol formed under the reaction conditions. Preferred solvents are water, THF, and NMP; particular preference is given to water.

The hydrogenation both in trickle and liquid phase mode, each preferably being carried out in circulation mode, is generally carried out at a temperature in the range from 50 to 350° C., preferably in the range from 70 to 300° C., more preferably in the range from 100 to 270° C., and a pressure in the range from 3 to 350 bar, preferably in the range from 5 to 330 bar, more preferably in the range from 10 to 300 bar.

In a very particularly preferred embodiment, the catalysts of the invention are employed in processes for preparing hexanediol and/or caprolactone, as described in DE 196 07 954, DE 196 07 955, DE 196 47 348 and DE 196 47 349.

The process according to the invention achieves high conversions and selectivities using the catalysts of the invention. At the same time, the catalysts of the invention have high chemical and mechanical stability.

The present invention therefore relates quite generally to the use of a treatment with boiling water and/or steam in the preparation of a catalyst to increase both the mechanical stability and the activity and selectivity of the catalyst.

In a preferred embodiment, the present invention relates to a use as described above, wherein the catalyst comprises copper as active component.

The mechanical stability of the solid-state catalysts and specifically of the catalysts of the invention is described by the side crushing strength parameter in various states (oxidic, reduced, reduced and suspended underwater).

The side crushing strength was determined for the purposes of the present application using an apparatus of the “Z 2.5/T 919” type supplied by Zwick Röll (Ulm). Both for the reduced and for the used catalysts, the measurements were carried out in methanol under nitrogen atmosphere in order to prevent reoxidation of the catalysts.

### EXAMPLES

### Example 1

### Preparation of Catalyst 1

A mixture of 12.41 kg of a 57% copper nitrate solution and 12.78 kg of a 33% aluminum nitrate solution and 0.48 kg of a 40% lanthanum nitrate.6H₂O solution was dissolved in 2 l of water (solution 1). Solution 2 contains 60 kg of a 20% anhydrous Na₂CO₃. Solution 1 and solution 2 were passed via separate lines into a precipitation vessel which is equipped with a stirrer and comprises 10 l of water heated to 80° C. In the course of this, the pH was brought to 6.2 by appropriate adjustment of the feed rates of solution 1 and solution 2.

While keeping the pH constant at 6.2 and the temperature at 60° C., the entire solution 1 was reacted with sodium carbonate. The suspension thus formed was subsequently stirred for a further 1 hour, in the course of which the pH is run at 7.2 by occasionally adding dilute nitric acid or soda solution 2. The suspension is filtered and washed with distilled water until the nitrate content of the washing water was <10 ppm.

The filtercake was dried at 120° C. for 16 h and subsequently calcined at 600° C. for 2 h. The catalyst powder thus obtained is precompacted with 1% by weight of graphite. The resulting compacted material is mixed with 5% by weight of Unicoat copper flakes and subsequently with 2% by weight of graphite and compressed to tablets of diameter 3 mm and height 3 mm. The tablets were finally calcined at 350° C. for 2 h.

The catalyst thus prepared has the the chemical composition 58% CuO/22% Al₂O₃/5% La₂O₃/15% Cu.

The side crushing strength was 25 N as specified in Table 1.

### Example 2

### Water Treatment for Catalyst 2

20 g of the catalyst according to Example 1 were mixed with 50 ml of water and heated at 140° C. and a pressure of 2 bar for 24 h. After the removal of the water, the catalyst was dried at 120 ° C. for 4 h.

### Example 3

### Steam Treatment for Catalyst 3

20 g of the catalyst according to Example 1 were treated at 140° C. at 1.3 bar with 100% steam for 20 h. The catalyst was then dried at 120° C. for 4 h.

### Example 4

Catalyst T4489 of composition 60% CuO/30% Al₂O₃/10 MnO₂, sold by Südchemie.

### Example 5

### Steam Treatment

The commercial catalyst of composition 60% CuO/30% Al₂O₃/10 MnO₂ (trade name T4489 from Südchemie) was treated with 100% steam at a pressure of 1.3 bar for 20 h and then dried at 120° C. for 4 h.

### Example 6

### Hydrogenation of Methyl Adipate Over Catalysts 1, 2, 3, 4 or 5

Dimethyl adipate was hydrogenated continuously in trickle mode with recycling (feed/recycle ratio=10/1) at an hourly space velocity of 0.3 kg/(l*h), a pressure of 200 bar and reaction temperatures of 210 bar and 190° C. in a vertical tubular reactor which had been charged in each case with 200 ml of catalysts 1, 2, 3, 4 or 5. The experimental duration was a total of 7 days. GC analysis detected, in the reactor effluent at 190° C., ester conversions of 99.9%, a hexanediol selectivity of 97.5%. After deinstallation, the catalyst was still fully preserved and had a high mechanical stability. The experimental results are compiled in Table 1.

The data in Table 1 which follows show that the inventive catalysts have significantly higher hydrogenation activities, i.e. higher conversions of dimethyl adipate at 190° C. than the comparative catalyst, and also higher product-of-value selectivities, i.e. contents of the hexanediol target products in the effluent.

TABLE 1

| | Reaction | Dimethyl adipate | Hexanediol | Side |
|---|---|---|---|---|
| Catalyst | temperature | conversion | selectivity | crushing |
| example | [° C.] | [%] | [%] | strength (N) |
| Catalyst 1 | 210 | 98.09 | 96.46 | 25 |
| (untreated) | | | | |
| Catalyst 2 | 210 | 99.57 | 97.34 | 54 |
| (water) | | | | |
| Catalyst 3 | 210 | 99.52 | 97.82 | 41 |
| (steam) | | | | |
| Catalyst 4 | 190 | 90.05 | 96.66 | 22 |
| (untreated) | | | | |
| Catalyst 5 | 190 | 92.8 | 97.1 | 34 |
| (steam) | | | | |

1. A process for hydrogenating an organic compound having at least one carbonyl group, in which the organic compound is contacted, in the presence of hydrogen, with a shaped body which is producible in a process in which
(i) an oxidic material comprising copper oxide and aluminum oxide and at least one of the oxides of iron, lanthanum, tungsten, molybdenum, titanium, zirconium, tin or manganese is provided,
(ii) pulverulent metallic copper, copper flakes, pulverulent cement, graphite or a mixture thereof are added to the oxidic material,
(iii) the mixture resulting from (ii) is shaped to a shaped body and
(iv) the shaped body is treated with boiling water and/or steam.

2. The process according to claim 1, wherein the oxidic material comprises
(a) copper oxide with a proportion in the range of 50≦x≦80% by weight,
(b) aluminum oxide with a proportion in the range of 15≦y≦35% by weight, and
(c) at least one of the oxides of iron, lanthanum, tungsten, molybdenum, titanium, zirconium, tin or manganese with a proportion in the range of 1≦z≦30% by weight,
based in each case on the total weight of the oxidic material after calcination, where: 80≦x+y+z≦100, cement not being included in the oxidic material in the above sense.

3. The process according to claim 1, wherein, by the addition, the pulverulent metallic copper, the copper flakes, the pulverulent cement or graphite or the mixture thereof is added in a proportion in the range of from 1 to 40% by weight based on the total weight of the oxidic material.

4. The process according to claim 1, wherein graphite is added to the oxidic material or to the mixture resulting from (ii) in a proportion in the range from 0.5 to 5% by weight based on the total weight of the oxidic material.

5. The process according to claim 1, wherein the shaped body is treated with boiling water at a pH of from 4 to 9.

6. The process according to claim 1, wherein the shaped body is treated with steam at from 1 to 20 bar and over 100° C.

7. The process according to claim 1, wherein the organic compound is a carboxylic acid, a carboxylic ester, a carboxylic anhydride or a lactone.

8. The process according to claim 7, wherein the organic compound is adipic acid or an adipic ester.

9. A shaped body treated with boiling water and/or steam and comprising
an oxidic material which comprises
(a) copper oxide with a proportion in the range of 50≦x≦80% by weight,
(b) aluminum oxide with a proportion in the range of 15≦y≦35% by weight, and
(c) at least one of the oxides of iron, lanthanum, tungsten, molybdenum, titanium, zirconium, tin or manganese with a proportion in the range of 1≦z≦30% by weight,
based in each case on the total weight of the oxidic material after calcination, where: 80≦x+y+z≦100,
metallic copper powder, copper flakes or cement powder or graphite or a mixture thereof with a proportion in the range of from 1 to 40% by weight, based on the total weight of the oxidic material, and
graphite with a proportion of from 0.5 to 5% by weight based on the total weight of the oxidic material,
where the sum of the proportions of oxidic material, metallic copper powder or cement powder or a mixture thereof and graphite add up to at least 95% by weight of the shaped body.

10. The method of treating a catalyst with boiling water and/or steam to increase both the mechanical stability and the activity and selectivity of the catalyst.

11. The method of treatment according to claim 10, wherein the catalyst comprises copper as an active component.
